(19) United States
(12) Patent Application Publication
(10) Pub. No.: US 2006/0035241 A1
(43) Pub. Date: Feb. 16, 2006

(54) Method for rapidly detecting quinolone-resistant Salmonella spp. and the probes and primers utilized therein

(75) Inventors: Shu-Jean Tsai, Taipei (TW); Shu-Wan Wang, Taipei (TW); Daniel Yang-Chih Shih, Taipei (TW)

Correspondence Address: BACON & THOMAS, PLLC, 625 SLATERS LANE, FOURTH FLOOR, ALEXANDRIA, VA, 22314, US

(73) Assignee: Bureau of Food and Drug Analysis, Department of Health, Executive Yuan, Taipei (TW)

(21) Appl. No.: 11/014,707

(22) Filed: Dec. 20, 2004

(30) Foreign Application Priority Data
Aug. 10, 2004 (TW) 93123850

(51) Int. Cl. C12Q 1/68

(52) U.S. Cl. 435/6

(57) ABSTRACT

The present invention relates to a method for rapidly detecting quinolone-resistant Salmonella spp. The invention also relates to the oligonucleotide primers and probes utilized in the method, which can differentiate mutant strains having one or two single point mutations in gyrA gene or single point mutation in parC gene from wild type strains, respectively. Said primers and probes can be effectively used for detection of nalidixic acid-resistant and/or ciprofloxacin-resistant Salmonella strains.

### BACKGROUND OF THE INVENTION

1. Field of the Invention

The invention relates to a method for rapidly detecting quinolone antibacterial-resistant Salmonella spp. and to oligonucleotide primers and probes utilized in said method.

2. Description of the Related Art

Recently, the problem of drug resistance of human and animal disease-causing microbes has been increasingly focused. Antibiotics and antibacterial agents are prevalently used for treatment and prophylaxis of human and animal diseases, which have resulted in the resistance of bacterial strains to the selective pressure of antibiotics and antibacterials in the environment. Furthermore, said substances are optionally used as feed additives to serve as animal growth enhancers, and thus the spread of drug-resistant bacterial strains are amplified (Cohen and Tauxe, 1986. Sci. 234:964-969; Liebana et al., 2002. J. Clinc. Microbiol. 40:1481-1486). As a result, increasing numbers of bacterial strains have showed resistance to the antibiotics and antibacterials which are commonly used for disease treatment. For example, such bacterial strains include multiple drug-resistant Salmonella typhimurium, methicillin-resistant Staphylococcus aureus) (MRSA), vancomycin-resistant enterococci, multiple drug-resistant Mycobacterium tuberculosis (Michel and Gutmann, 1997. Lancet 349:1901-1906; Moss et al., 1997. Int. J. Tuberc. Lung Dis. 1:115-121).

A third generation quinolone antibacterial agents, fluoroquinolones, have been developed since 1988 and can effectively treat bacterial infection in intestines. However, after enrofloxacin was approved to be used as drugs for animal therapy in 1993, multiple drug-resistant Salmonella DT104 isolated from human and animal specimens was mostly found to have not only resistance to nalidixic acid but also decreased susceptibility to ciprofloxacin, one of fluoroquinolone antibiotics (Piddock et al., 1993. Antimicrob. Agents Chemother. 37:662-666; Walker et al., 2001. J. Clinc. Microbiol. 39-1443-1448; Wiuff et al., 2000. Microb. Drug Res. 6:11-17.). Prior researches have showed that the therapeutic effects of fluoroquinolone antibacterial agents on the infections caused by Salmonella were reduced due to the bacterial resistance to nalidixic acid (McCarron and Love, 1997. Clinc. Infect. Dis. 24:707-709; Vasallo et al., 1998. Clinc. Infect. Dis. 26:535-536). Furthermore, when the susceptibility of bacterial strains to fluoroquinolones is reduced, standard dosages of said drugs for treatment may not be sufficient to treat clinical infections. However, under the selective environmental pressure in the presence of an antibacterial agent, bacterial strains having resistance to ciprofloxacin can beneficially survive and grow, resulting in significant increased numbers of strains having resistance to ciprofloxacin and eventually leading to treatment failures. As a result, the emergence of Salmonella strains resistant to ciprofloxacin has become a serious problem in clinical treatment (Pers et al., 1996. Scand. J. Infect. Dis. 28:529-531; Piddock et al., 1990. Lanct 335:1459; Piddock et al., 1993. Antimicrob. Agents Chemother. 37:662-666; Vasallo et al., 1998. Clinc. Infect. Dis. 26:535-536; Wain et al., 1997. Clinc. Infect. Dis. 25:1404-1410).

Specifically, Salmonella spp. isolated from clinical specimens and animal foods and resistant to quinolone antibacterials have been gradually increased in recent years (Davies et al., 1999. Vet. Rec. 20:320-322; Herikstad et al., 1997. Emerg. Infect. Dis. 3:371-372; Malomy et al., 1999. Antimicro. Agents Chemother. 43:2278-2282; Wiuff et al., 2000. Microb. Drug Res. 6:11-17). According to the surveillance results of Veterinary Laboratory Agency (VLA) in 1999, about 5.3% of all Salmonella strains isolated from animals were resistant to nalidixic acid. Among the isolates, poultry isolates showed the highest percentage of resistance, with 13.4% of the isolates having resistance to nalidixic acid (Evan and Kidd, 1999. Veterinary Laboratories Agency, Ministry of Agriculture, Fisheries and Food, London, United Kingdom. p. 127-133). According to the monitoring results of the drug resistance of Salmonella spp. isolated from animals by National Antimicrobial Resistance Monitoring System (NARMS) from 1997 to 2002, the percentage of the bacteria having resistance to nalidixic acid ranged from 0.8 to 2.0%. Among said resistant bacteria, those isolated from turkeys showed the highest percentage of 2.1-5.5%, while all bacteria were susceptible to ciprofloxacin (USDA/ARS, 2002). During the continuous monitoring the drug resistance of Salmonella isolated from the clinical specimens from 1996 to 2001, the percentage of the bacteria resistant to nalidixic acid ranged from 0.4 to 3% and those resistant to ciprofloxacin ranged from 0 to 0.4%, while the bacteria having decreased resistance (MICs≧0.25 μg/mL) ranged from 0.4 to 1% (CDC, 2003). The monitoring results showed that the percentage of Salmonella either from animal or clinical sources which have resistance to nalidixic acid has been gradually increased.

It is known that the causes for drug resistance of bacteria to quinolone antibacterials include: mutations in the structural genes of bacterial gyrase and topoisomerase IV as the target enzymes of quinolone antibacterials for bacterial inhibition, a decrease in drug permeability of bacterial cell membranes to said type of antibacterials, and change in an active efflux mechanism of bacteria (Cambau and Gutmann, 1993. Drugs 45 (Suppl. 3):15-23; Hooper et al., 1992. Antimicrob. Agents Chemother. 36:1151-1154; Poole, 2000. Antimicrob. Agents Chemother. 44:2233-2241). In the above different mechanisms, amino acid substitutions in the Gyr subunit resulted from mutations in gyrA play a considerably critical role in the resistance of gram-negative (G(−)) bacteria to quinolone antibacterials (Hooper, 1999. Drug Resist. Updates 2:38-55; Nakamura, 1997. J. Infect. Chemother. 3:128-138; Piddock, 1999. Drugs 58 (Suppl. 2):11-18). High-level resistance of Salmonella to quinolones has been believed to be associated with point mutations in quinolone resistance-determining region (QRDR) of gyrA (the translated amino acids thereof are between positions 67 and 122). The mutations can be Ser83→Phe (TCC→TTC), Ser83→Tyr (TCC TAC), Asp87→Gly (GAC→GGC), Asp87→Asn (GAC→AAC) and Asp87→Tyr (GAC→TAC) (Cambau and Gutmann, 1993. Drugs 45 (Suppl. 3):15-23; Grigg et al., 1996. Antimicrob. Agents Chemother. 40:1009-1013; Heising et al., 1995. Microb. Drug Resist. 1:211-218; Heurtin et al., 1999. J. Clin. Microbiol. 37:266-269; Walker et al., 2001. J. Clinc. Microbiol. 39-1443-144; Piddock et al., 1998. J. Antimicrob. Chemother. 41:635-641; Wiuff et al., 2000. Microb. Drug Res. 6:11-17). In addition to the above five mutations in gyrA, mutation at codon 119 Ala→Val may also be correlated with the resistance of one strain of Salmonella typhimurium to nalidixic acid (Griggs et al., 1996. Antimicrob. Agents Chemother. 40:1009-1013). Nevertheless, in bacterial strains having resistance to quinolone antibacterials and reduced susceptibility which are most commonly detected from clinical samples, mutations in the gyrA genes primarily occur on the positions of Ser83 and Asp87. Furthermore, point mutations in the other related genes, gyrB, parC and parE, may also increase the resistance of the strains to fluoroquinolones (Giraud et al., 1999. Antimicrob. Agents Chemother. 43:2131-2137; Piddock et al., 1998. J. Antimicrob. Chemother. 41:635-641; Wiuffet al., 2000. Microb. Drug Res. 6:11-17). However, said point mutations have not yet been found in bacterial strains resistant to only nalidixic acid (Reyna et al., 1995). Point mutations in genes can be identified using the methods such as single nucleotide polymorphism (SNP), restriction fragment length polymorphism (RFLP), single-stranded conformational polymorphism (SSC), mismatch polymerase chain reaction (mismatch PCR) and sequence analysis (Qiang et al., 2002. J. Antimicrob. Chemother. 49:549-552; Quabdesselam et al., 1995. Antimicrob. Agents Chemother. 39:1667-1670; Vacher et al., 2003. Antimicrob. Agents Chemother. 47:1125-1128; Zimstein et al., 1999. J. Clin. Microbiol. 37:3276-3280). In the SNP detection method, real-time PCR method using TaqMan probes, hybridization probes or SYBR Green has been developed in the recent years. Said method has been applied to rapidly detect the polymorphism of human genes which induces sensitivity of human bodies to drugs, for genotyping of bacteria having resistance to quinolone antibacterials, etc. (Brooks, 1999. Gene 234:177-186; Hiratsuka et al., 2002. Clin. Biochem. 35:35-40; Mhlanga and Malmberg, 2001. Methods 25:463-471).

For SNP analysis using real-time PCR, a specific single-stranded PCR product has to be hybridized with hybridization probes, followed by the performance of melting curve analysis (Carattoli et al., 2002. FEMS Microbiol. Lett. 214:87-93; Hiratsuka et al., 2002. Clin. Biochem. 35:35-40; Caplin et al., 1999. Biochemica 1:5-9; Lindler et al., 2001. J. Clinc. Mcirobiol. 39:3649-3655; Lareu et al., 2001. Forensic Sci. Int. 118:163-168). When the LightCycler system is used, two oligonucleotide probes labeled with light-activated molecules positioned in close proximity can be utilized, in which one probe contains fluorescent LC-Red640 or LC-Red705 label and the other adjacent labeled probe assists to produce fluorescence by fluorescence resonance energy transfer (FRET). Namely, the two probes can bind to the specific region in a single-stranded amplified product (amplicon), wherein one probe is used as an anchor probe which can intimately bind to a single-stranded PCR amplification product, and the other adjacent probe is used as a sensor probe or mutation probe which binds to the variable region of the amplified product. When the two labeled probes both specifically bind to the single-stranded PCR amplification product and are considerably close to each other, energy is transferred to LC-Red640 or LC-Red750 by FRET to generate fluorescence emission. Therefore, fluorescence increases as more PCR product accumulates, whereby the PCR amplification product can be quantified. Additionally, while DNA melting point analysis is performed and the temperature is gradually increased, the close binding state between the probes and single-stranded PCR product tends to become an unstable binding state which results in fluorescence decrease. If the degree of mismatch between the sensor probe and the amplification product increases, the binding stability between them would be reduced and the detected Tm would be lowered. By the Tm change between the sensor probe and single-stranded PCR product, the sequence differences of the detected target genes can be speculated. Taking a double-stranded chromosome as an example, a single Tm peak is obtained if present as the same wild type; two peaks can be detected if present as mix-alleles; and a single peak different from that of a wild type can be detected if present as the same type of mutants. In addition, a temperature shift resulting from mismatch of a single mutation can display 5 to 9° C. of Tm change. As a result, detection of point mutations in genes by applying this method shows considerable sensitivity (Caplin et al., 1999. Biochemica 1:5-9; Hiratsuka et al., 2002. Clin. Biochem. 35:35-40).

A combination of real-time PCR and melting curve analysis using fluorescent labeled probes can avoid the analysis steps required by traditional gel electrophoresis. Said method exhibits the advantages of rapidity, sensitivity and specificity and has been applied to largely and rapidly screen bacterial strains for point mutations in genes. For example, this method has been developed for identification of multiple drug resistant Salmonella spp. having resistance to nalidixic acid and decreased susceptibility to ciprofloxacin, and Campylobacter jejuni and C. coli having resistance to ciprofloxacin (Carattoli et al., 2002. FEMS Microbiol. Lett. 214:87-93; Liebana et al., 2002. J. Clinc. Microbiol. 40:1481-1486; Walker et al., 2000. Vet. Rec. 147:395-396; Walker et al., 2001. J. Clinc. Microbiol. 39-1443-1448; Wilson et al., 2000. J. Clinc. Microbiol. 38:3971-3978). Nevertheless, for the detection of gyrA mutation in Salmonella strains, the mutation types are various and mostly are the point mutations of nucleotide G to A or T, or the point mutations of nucleotide C to A or T. As the structural stability of mismatch between G or C and A and the structural stability of mismatch between G or C and T are relatively similar, mutations are difficult to be distinguished. Furthermore, the presence of the secondary structure of a stem loop near the mutation position in QRDR of gyrA in Salmonella strains also interferes the pattern of melting curve analysis (Caplin et al., 1999. Biochemica 1:5-9). Accordingly, up to date, the reports of the research pertaining to real-time PCR combined with melting curve analysis have not been capable of effectively distinguishing or identifying mutants having different types of gyrA point mutations (Liebana et al., 2002. J. Clinc. Microbiol. 40:1481-1486; Walker et al., 2000. Vet Rec. 147:395-396; Walker et al., 2001. J. Clinc. Microbiol. 39-1443-1448). Furthermore, no related reference or report has published the application of real-time PCR and the use of hybridization probes in combination with the performance of melting curve analysis to rapidly detect or identify ciprofloxacin-resistant Salmonella strains having point mutations in the parC gene.

Generally, a traditional detection of Salmonella resistance to quinolone antibacterials commonly utilizes serial dilution or filter diffusion. Said methods require preparation of media, adjustment of the amount of tested bacteria and incubation time of 18 to 24 hours, followed by determination based on the growth condition of tested bacteria and the diameter of bacterial inhibition zone. Said methods involve redundant procedures of experiments and are time-consuming. As a result, there is a need to develop rapid and precise methods for detection of quinolone-resistant Salmonella spp.

### BRIEF SUMMARY OF THE INVENTION

The invention relates to a method for rapidly detecting quinolone antibacterial-resistant Salmonella spp. The invention also relates to oligonucleotide primers and probes utilized in said method, which can be used to rapidly distinguish wild type strains from mutant strains having single or double point mutations in gyrA or mutants having single point mutation in parC, respectively, thereby enabling the detection of Salmonella strains having resistance to nalidixic acid and/or ciprofloxacin.

### BRIEF DESCRIPTION OF THE DRAWINGS

FIG. 1 is a graph of melting curve analysis of hybridization probe set gyrA4 hybridized with single-stranded PCR products of gyrA.

FIG. 2 is a graph of melting curve analysis of hybridization probe set parC238 hybridized with single-stranded PCR products of parC.

### DETAILED DESCRIPTION OF THE INVENTION

The present invention is directed to the sets of primers and probes designed based on the gyrA and parC genes in different serotypes of Salmonella strains, in order to establish a method for rapidly detecting and identifying Salmonella strains resistant to quinonlone antibacterials.

In one aspect of the invention, the present invention provides a method for rapidly detecting Salmonella spp. resistant to quinolone antibacterials, which comprises amplification of Salmonella DNA by polymerase chain reaction (PCR) in a sample to be tested using a specific primer set, hybridization of a specific probe set with the single-stranded PCR product, performing melting curve analysis and analyzing the Tm change of the hybrid of the single-stranded PCR product with the hybridization probes, thereby differentiating wild-type Salmonella strains from mutants resistant to quinolones.

In one embodiment, the present invention provides a method for rapidly detecting Salmonella spp. resistant to quinolone antibacterials, which comprises the steps of:
(a) isolating Salmonella DNA from a sample to be detected;
(b) amplifying the Salmonella DNA obtained from step (a) by polymerase chain reaction (PCR) using a primer set derived from the nucleotide sequences of the gyrA or parC gene;
(c) hybridizing a probe set based on the nucleotide sequences of the gyrA or parC gene with the single-stranded PCR product obtained from step (b); and
(d) performing melting curve analysis to analyze the Tm change of the hybrid of the single-stranded PCR product with the hybridization probes, thereby distinguishing wild-type Salmonella strains from mutants resistant to quinolone antibacterials.

In the method of the invention, the probe sets used are designed based on the detection targets, nucleotides 248, 259 and 260 of gyrA and nucleotide 238 of parC at which point mutations occur. Said probes can rapidly detect Salmonella strains resistant to nalidixic acid and/or ciprofloxacin.

In the method of the present invention, the primer sets used include gyrA55/gyrA330 with the nucleotide sequences of SEQ ID NOS: 1 and 2 and parC202/parC348 with the nucleotide sequences of SEQ ID NOS:3 and 4. The probe sets used include the gyrA4 probe set with the nucleotide sequences of SEQ ID NOS: 5 and 6 and the parC238 probe set with the nucleotide sequences of SEQ ID NOS: 7 and 8. Among the primer sets, the primer set gyrA55/gyrA330 (SEQ ID NOS:1 and 2) is used in combination with the gyrA4 probe set (SEQ ID NOS:5 and 6) designed based on gyrA mutational properties to distinguish wild-type Salmonella strains from mutants having single or double point mutations in gyrA, thereby rapidly detecting Salmonella strains resistant to nalidixic acid and/or ciprofloxacin. In addition, the primer set parC202/parC348 (SEQ ID NOS:3 and 4) is used in combination with the parC238 probe set (SEQ ID NOS:7 and 8) designed based on parC mutational properties to distinguish wild-type Salmonella strains from mutants having single point mutations in parC, thereby rapidly detecting Salmonella strains resistant to ciprofloxacin.

In the method of the present invention, quinolone antibacterial agents include nalidixic acid and ciprofloxacin. In step (c), the probe set utilized in hybridization can specifically bind to the single-stranded PCR amplification product. Said probe set is used in combination with fluorescent labels, in which one probe is labeled with fluorescent LC-Red640 and the other probe assists to produce fluorescence by fluorescence resonance energy transfer (FRET), whereby the PCR amplification product can be quantified.

In a preferred embodiment of the method of the present invention, the invention relates to a method for rapidly detecting Salmonella spp. resistant to nalidixic acid and/or ciprofloxacin, which comprises the steps of:
(a) isolating Salmonella DNA from a sample to be detected;
(b) amplifying the Salmonella DNA obtained from step (a) by polymerase chain reaction (PCR) using the primer set gyrA55/gyrA330 (SEQ ID NOS:1 and 2);
(c) hybridizing the probe set gyrA4 (SEQ ID NOS:5 and 6) with the single-stranded PCR product obtained from step (b); and
(d) performing melting curve analysis to analyze the Tm change of the hybrid of the single-stranded PCR product with the hybridization probes, thereby distinguishing wild-type Salmonella strains from mutants having single or double point mutations in gyrA.

In another preferred embodiment of the method of the present invention, the invention relates to a method for rapidly detecting Salmonella spp. resistant to ciprofloxacin, which comprises the steps of:
(a) isolating Salmonella DNA from a sample to be detected;
(b) amplifying the Salmonella DNA obtained from step (a) by polymerase chain reaction (PCR) using the primer set parC202/parC348 (SEQ ID NOS:3 and 4);
(c) hybridizing the probe set parC238 (SEQ ID NOS:7 and 8) with the single-stranded PCR product obtained from step (b); and

(d) performing melting curve analysis to analyze the Tm change of the hybrid of the single-stranded PCR product with the hybridization probes, thereby distinguishing wild-type Salmonella strains from mutants having single point mutations in parC.

In a second aspect of the present invention, the present invention relates to an oligonucleotide primer set, wherein the first primer has the nucleotide sequence AGC TCC TAT CTG GAT TAT GC (gyrA55, SEQ ID NO:1) and the second primer has the nucleotide sequence ACC GAA GTT ACC CTG AC (gyrA330, SEQ ID NO:2). Said primer set is used for amplifying the DNA sequence of the target region of Salmonella gyrA or the complementary strand thereof, which can distinguish wild-type Salmonella strains from mutants having single or double point mutations in gyrA and thereby differentiating wild-type Salmonella strains from mutants resistant to nalidixic acid and/or ciprofloxacin.

Furthermore, the present invention relates to another oligonucleotide primer set, wherein the first primer has the nucleotide sequence GGT GAC GTA CTG GGT A (parC202, SEQ ID NO:3) and the second primer has the nucleotide sequence CGC GAA TGA CTT CGG A (parC348, SEQ ID NO:4). Said primer set is used for amplifying the DNA sequence of the target region of Salmonella parC or the complementary strand thereof, which can distinguish wild-type Salmonella strains from mutants having single point mutations in parC and thereby differentiating wild-type Salmonella strains from mutants resistant to ciprofloxacin.

Moreover, in a third aspect of the present invention, the invention provides an oligonucleotide probe set, wherein the first probe has the nucleotide sequence CGA TTC CGC AGT GTA TGA CAC C-FL (gyrA4-FL, SEQ ID NO:5) and the second probe has the nucleotide sequence LCRED640-CGT TCG TAT GGC GCA GCC ATT CTC G-PHO (gyrA4-LC640, SEQ ID NO:6). Said probe set can hybridize with the DNA sequence of the target region of Salmonella gyrA or the complementary strand thereof, which is used to distinguish wild-type Salmonella strains from mutants having single or double point mutations in gyrA and thereby detecting Salmonella mutant strains resistant to nalidixic acid and/or ciprofloxacin. Furthermore, the present invention relates to another oligonucleotide probe set, wherein the first probe has the nucleotide sequence CGA CCG CGC CTG CT-FL (parC238-FL, SEQ ID NO:7) and the second probe has the nucleotide sequence LCRED640-GAA GCC ATG GTG CTG ATG GCG-PHO (parC238-LC640, SEQ ID NO:8). Said probe set can hybridize with the target region of Salmonella parC or the complementary strand thereof, which can distinguish wild-type Salmonella strains from mutants having single point mutations in parC and thereby detect Salmonella mutant strains resistant to ciprofloxacin.

The method of the present invention utilizes novel sets of primers and probes in combination with real-time polymerase chain reaction and melting curve analysis to determine the Tm of the hybrid of the single-stranded PCR product of gyrA or parC with the hybridization probes. Said method can detect the resistance of Salmonella spp. to quinolone antibacterials in one hour, which significantly reduces the time required by traditional detection methods. The method is simple, rapid and precise, which can be used to screen large numbers of bacterial isolates and thus has beneficial effects in practical clinical applications. Furthermore, the probe sets utilized in the method of the present invention can also be used for genotyping and therefore can be used for epidemiology research such as effective tracing the source of contamination.

### EXAMPLES

The following examples are used to illustrate the technical content of the present invention and the efficacy to be achieved, but not used to limit the present invention. Any equivalent changes and modifications made according to the invention are all within the scope of the claims of the invention.

Tested Bacterial Strains

Eighty strains isolated from commercial animal products in 1999 and from 2001 to 2003, twenty-eight strains isolated from clinical samples in 2001, three strains provided by Food and Drug Administration (FDA), USA, and nine strains purchased from Bioresources Collection and Research Center of Food Industry and Research Development Institute in Hsinchu, Taiwan are used. There are thirty-two serotypes of 120 Salmonella strains in total, including 33 strains of S. schwarzengrund having resistance or susceptibility to quinolone antibacterials, 20 strains of S. typhimurium, 11 strains of S. enteritidis, 10 strains of S. albany, 4 strains of S. derby, 3 strains of each of S. agona and S. istanbul, 2 strains of each of S. anatum, S. hardt, S. II, S. livingstone, S. london, S. murnvhen, S. newport, S. paratyphi B, S. senftenberg, S. stanley and S. typhi, and one strain of each of S. amsterdam, S. bovismorbificians, S. dublin, S. choleraesuis, S. gombe, S. havana, S. haifa, S. mbandaka, S. panama, S. paratyphi A, S. weltevreden, S. reading, S. virchow and Salmonella spp. (O4:Hi:1,7).

Primers and Probes

The primer and probe sets used in the method of the present invention include the primer set gyrA55/gyrA330 and the probe set gyrA4 as well as the primer set parC202/parC348 and the probe set parC238, the nucleotide sequences of which are shown in Table 1.

TABLE 1
The primer and probe sequences used in the present
invention

| | | |
|---|---|---|
| Name | sequence (5′ → 3′) | SEQ ID NO |

Primer

| | | |
|---|---|---|
| gyrA55/gyrA330 | | |
| gyrA55 | AGC TCC TAT CTG GAT TAT | SEQ ID NO:1 |
| | GC | |
| gyrA330 | ACC GAA GTT ACC CTG AC | SEQ ID NO:2 |
| parC202/ | | |
| parC348 | | |
| parC202 | GGT GAC GTA CTG GGT A | SEQ ID NO:3 |
| parC348 | CGC GAA TGA CTT CGG A | SEQ ID NO:4 |
| Probe | | |
| gyrA4 | | |
| gyrA4-FL | CGA TTC CGC AGT GTA TGA | SEQ ID NO:5 |
| | CAC C-FL | |
| gyrA4-LC640 | LCRED640-CGT TCG TAT GGC | SEQ ID NO:6 |
| | GCA GCC ATT CTC G-PHO | |
| parC238 | | |
| parG238-FL | CGA CCG CGC CTG CT-FL | SEQ ID NO:7 |
| parC238-LC640 | LCRED640-GAA GCC ATG GTG | SEQ ID NO:8 |
| | CTG ATG GCG-PHO | |

### Example 1

Detection of Resistance of Salmonella spp. to Quinolone Antibacterials and of Gene Mutations Therein

Determination of Minimum Inhibitory Concentration (MIC) of Quinolone Antibacterials

The minimum inhibitory concentration was determined by the E-test strips of quinolone antibacterials (AB BIODISC North America Inc., Solna, Sweden). The bacterial strain to be tested was streak plated on xylose lysin desoxycholate agar (XLD) (Merck kGaA, Darmstadt, Germany). After the purity of said strain was verified, the strain was grown on tryptic soy agar (TSA) medium (Merck kGaA, Darmstadt, Germany) at 35-37° C. for 16-18 hours and then suspended in sterile water. The bacterial concentration was adjusted to 80% of turbidity (corresponding to 0.5 MacFarland turbidity standard, and the bacterial content is approximately 10⁷ CFU/ml) using Vitek Special DR 100 Colorimeter (HACH Company, Colorado, USA). Then, 0.1 ml of the bacterial aliquot was placed on the prepared Mueller-Hinton (M-H) agar medium (having the diameter of 9 cm and the thickness of 4.0±0.5 mm) (Merck kGaA, Darmstadt, Germany) and the aliquot was spread evenly on the medium by a sterilized glass rod. The medium plate stood still for 10 to 15 minutes to ensure that the bacterial aliquot completely penetrated into the medium. Thereafter, a sterilized tweezer was used to clip the E region of the E-test antibacterial test strip which was then placed on the inoculated M-H medium plate. Two E-test trips were placed parallel to each other and symmetrically on each medium plate. Said medium plates were then incubated at 35-37° C. for 16-18 hours. The results were determined based on the standards from National Committee for Clinical Laboratory Standard (NCCLS, 2001), USA. Each antibacterial agent was tested in a duplicate experiment, and E. coli ATCC 25922/CCRC 11509 was used as the control of the tested strains in each experiment. The detection range of the antibacterial agent, nalidixic acid, is from 0.016-256 μg/ml, and the detection range of ciprofloxacin is from 0.002-32 μg/ml.

Detection of the gyrA and parC Genes in Salmonella spp.

1. PCR Amplification

The strain to be tested was streak plated on the XLD. After the purity of said strain is verified, the bacterial strain was incubated in the TSB broth (Merck kGaA, Darmstadt, Germany) at 35±1° C. for 18±2 hours. After refrigerated centrifugation, the genomic DNA of the tested bacterial strain was isolated with Wizard® Genomic DNA Purification Kit (Promega Co., Madison, Wis., USA). One microliter of the aliquot from DNA extraction was added to the PCR reaction mix (containing 20 μM DATP, 20 μM dTTP, 20 μM dCTP, 20 μM dGTP, one Unit of Taq DNA polymerase and 1 μM of each of the two primer sets shown in Table 1) to a final volume of 50 μl, which was placed in GeneAmp PCR System 9700 (Applied Biosystems, Inc., California, USA) to carry out PCR amplification. In said PCR reaction, the cycling conditions of amplification are: total 35 cycles of 94° C. for 1 minute, 60° C. for 1 minute and 72° C. for 2 minutes, using the primers with the nucleotide sequences shown in Table 2.

After amplification was completed, 10 μl of the reactant was mixed with 2 μl of a gel-loading buffer containing 0.25% bromophenol blue, 0.25% xylene cyanol FF and 15 Ficoll (Type 400, Pharmacia) dissolved in water and then was loaded onto the wells of 2% electrophoresis agarose gel. Electrophoresis was conducted in 0.5×TBE buffer (1× solution containing 0.089 M tris, 0.089 M borate and 0.002 M EDTA, made by dilution with 5× TBE buffer) for about 40 to 45 minutes. The electrophoresis gel was stained with 0.5 μg/ml ethidium bromide solution for 30 minutes and then destained with clean water for about 20 minutes. The gel was observed and photographed at 312 nm UV wavelength in an ultraviolet (UV) box (Image Master VDS, Pharmacia Biotech, Inc., USA). To assure the accuracy of the experiment, S. typhimurium DT 104 FDA 4 was used as a control strain in each processing of PCR amplification.

TABLE 2
SNP condition used in the present invention

| | | Target | Hold time | Slope | Acquisition |
|---|---|---|---|---|---|
| Primers/Probes | Program | temp. (° C.) | (° C.) | (° C./s) | mode |
| gyrA55/330 | Amplification^{a} | | | | |
| gyrA4 LC640/FL | 1 | 95 | 10 | 20 | None |
| (gyrA4) | 2 | 58 | 20 | 20 | Single |
| | 3 | 72 | 20 | 20 | None |
| | Melting^{b} | | | | |
| | 1 | 95 | 10 | 20 | None |
| | 2 | 35 | 60 | 20 | None |
| | 3 | 95 | 0 | 0.1 | Continuous |
| parC202/348 | Amplification | | | | |
| parC238 LC640/FL | 1 | 95 | 10 | 20 | None |
| (parC238) | 2 | 60 | 25 | 20 | Single |
| | 3 | 72 | 15 | 20 | None |
| | Melting | | | | |
| | 1 | 95 | 10 | 20 | None |
| | 2 | 35 | 30 | 20 | None |
| | 3 | 95 | 0 | 0.1 | Continuous |

^{a}1: denaturation, 2: annealing, 3: extension
^{b}1: denaturation, 2: hybridization, 3: melting curve analysis

2. Sequencing and Sequence Alignment

The sequencing of the product yielded by PCR amplification was carried out in a sequencer CEQ 2000 XL (Beckman Coulter Co., California, USA) based on Sanger method. The sequences were analyzed using the GCG Command Mode analysis-alignment program (Wisconsin Package Version 10.2) provided by National Health Institute, Executive Yuan, Taiwan.

Results

As shown in Table 3, the resistance of the tested 120 Salmonella strains isolated from animal foods and clinical samples to nalidixic acid and the gyrA genes therein were detected. Among the bacterial strains, both of codons 83 and 87 in gyra QRDR in the strains susceptible to nalidixic acid (i.e. wild type) (MICs 2.5-7 μg/ml) did not change and were of 248C/259G/260A type. There were 45 strains in which the amino acids translated from the two codons remained Ser83 and Asp87, corresponding to 37.5% of all strains. Seventy-five strains (62.5% of all strains) were resistant to nalidixic acid (i.e. mutants) (MICs≧256 μg/ml). Based on the mutation positions on codons 83 and 87 of gyrA QRDR and the amino acids translated therefrom, there were five types of mutations. The first type, 14 strains (11.7%) in total, is G to A transition at nucleotide 259 which belongs to G259A mutation type resulting in the respective translated amino acids Ser83 and Asn87. The second type, two strains (1.7%) in total, is G to T transversion at nucleotide 259 which belongs to G259T mutation type, and the translated amino acids are Ser83 and Tyr87, respectively. The third type, 11 strains (9.2%) in total, is C to A transversion at nucleotide 248 which belongs to C248A mutation type, and the translated amino acids are Tyr83 and Asp87, respectively. The fourth type, 25 strains (20.8%) in total, is C to T transition at nucleotide 248 which belongs to C248T mutation type, and the translated amino acids are Phe83 and Asp87. The fifth type, 23 strains (19.2%) in total, is C to T transition at nucleotide 248 and A to G transition at nucleotide 260 which belongs to C248T/A260G mutation type, and the translated amino acids are Phe83 and Gly87. Furthermore, among the 120 Salmonella strains, point mutations also occur in the parC genes of 23 strains (19.2%) with C248T/A280G mutations.

TABLE 3
The test results of the resistance of the detected 120 Salmonella strains to quinolone
antibacterial agents and the mutation types of the gyrA and parC genes in the strains

| | | | | gyrA | | | GyrA | | |
|---|---|---|---|---|---|---|---|---|---|
| | Number of strain | Susceptibility | | Mutation point | | | amino acid | | parC Mutation point |
| Strain type | (%) | NA^{a} | Cp^{b} | 248^{c} | 259 | 260 | 83^{d} | 87 | 238^{c} |
| Wild type | 45 (37.5) | S | S | C | G | A | Ser | Asp | A |
| Mutant | 75 (62.5) | R | S/R | | | | | | |
| G259A | 14 (11.7) | R | S | C | A | A | Ser^{e} | Asn | A |
| G259T | 2 (1.7) | R | S | C | T | A | Ser | Tyr | A |
| C248A | 11 (9.2) | R | S | A | G | A | Tyr | Asp | A |
| C248T | 25 (20.8) | R | S | T | G | A | Phe | Asp | A |
| C248T/A260G | 23 (19.2) | R | R | T | G | G | Phe | Gly | C |

^{a}NA, nalidixic acid; S, susceptibility, MICs 2.5˜7 μg/ml; R, resistance, MICs ≧256 μg/ml
^{b}Cp, ciprofloxacin; S, susceptibility, MICs 0.006-0.38 μg/ml; R, resistance, MICs 4˜≧32 μg/ml
^{c}position of nucleotide
^{d}position of amino acid
^{e}Ser, serine; Asn, asparagine; Tyr, tyrosine; Asp, aspartic acid; Phe, phenylalanine; Gly, glycine

### Example 2

Detection of gyrA and parC using Real-Time PCR Combined with Melting Curve Analysis

The bacterial strain to be tested was streak plated on XLD medium. After the purity of said strain was verified, the strain was grown in TSB broth at 35±1° C. for 18±2 hours. Following refrigerated centrifugation, Wizard® Genomic DNA Purification Kit was used to extract the genomic DNA. One microliter of the DNA extraction solution was added to the reaction mix (containing 0.5 μM primer set, 0.2 μM probe set, 4 mM MgCl₂, and 1× LightCycler DNA Master hybridization mix) to a final volume of 20 μl, which was then placed in LightCycler (Roche Applied Science, Mannheim, Germany) for amplification and melting reaction of the hybrid of the PCR product with the hybridization probes. The conditions of the amplification and melting reactions are shown in Table 2.

Results

The gyrA-specifc primer set gyrA55/gyrA330 in combination with the gyrA4 hybridization probe sets (including probes gyrA4-FL and gyrA4-LC640) were used for detection of 45 wild-type Salmonella strains and 75 mutant strains with five different types of mutations in Example 1. Following amplification, a 276 bp PCR product is obtained. The gyrA4 probe set was hybridized with the single strand of said product, and the melting curve analysis (Tm) of the resulted hybrid was carried out. The Tms of the wild-type strains are 63.6±1.0° C. (CV % of 1.62); the Tms of the mutant strains belonging to G259A mutation type are 59.8±0.4° C. (CV % of 0.60); the Ts of the mutant strains belonging to G259T mutation type are 59.6±0.4° C. (CV % of 0.68); the Tms of the mutant strains belonging to C248A mutation type are 58.6±0.3° C. (CV % of 0.51); the Tms of the mutant strains belonging to C248T mutation type are 59.1±0.2° C. (CV % of 0.40); and the Tms of the mutant strains belonging to C248T/A260G mutation type are 54.6±0.2° C. (CV % of 0.27), as shown in Table 4.

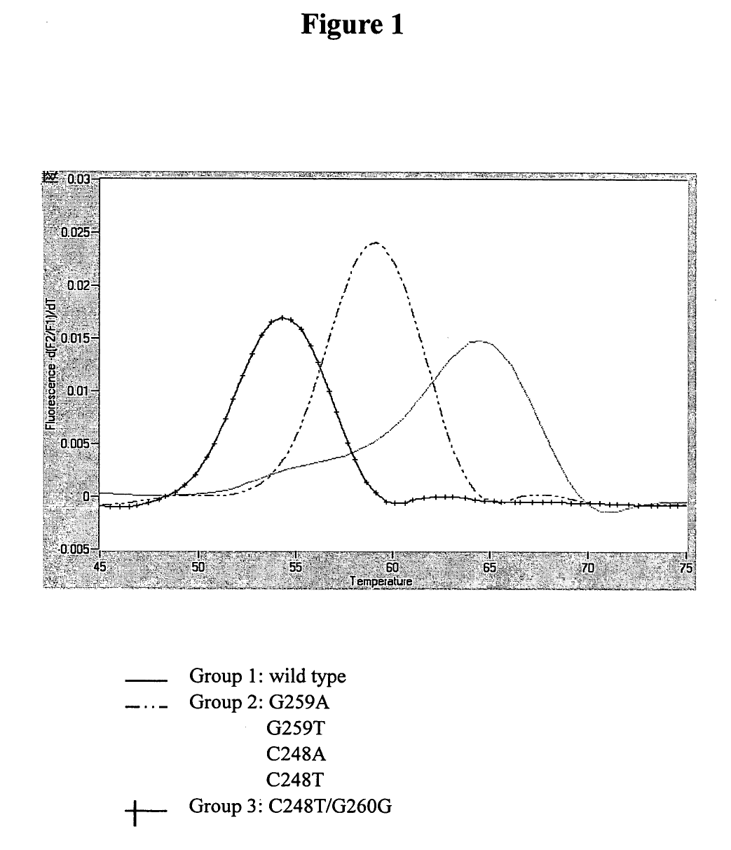

All tested 120 Salmonella strains can be classified into three groups based on Tms using the gyrA4 probe set. The first group includes 45 strains (corresponding to 37.5% of all strains) in which no mutation occurred in the QRDR region of the gyrA gene, which has the Tm of 63.6±1.0° C. (CV % of 1.62). The second group includes 52 strains (corresponding to 43.3% of all strains) in which a single point substitution occurred at nucleotide 248 or 259, including the mutants belonging to C248A, C248T, G259A and G259T mutation types, which has the Tm of 59.2±0.5° C. (CV % of 0.89). The third group includes 23 strains (corresponding to 19.2% of all strains) in which mutations occur at both nucleotides 248 and 260, belonging to C248T/A260G mutation type, which has the Tm of 54.6±0.2° C. (CV % of 0.27), as shown in Table 4 and FIG. 1. The results showed that the gyrA4 probe set can rapidly detect wild-type Salmonella strains and mutants having a single point mutation and double point mutation in the gyrA gene.

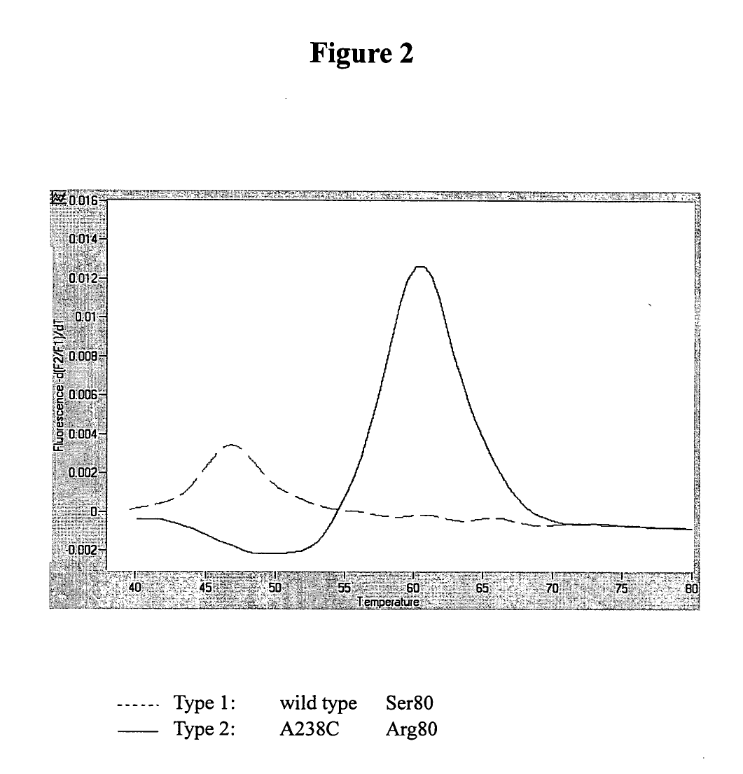

Furthermore, among the 120 Salmonella strains tested, 97 strains (80.8%) showed susceptibility to ciprofloxacin, in which no mutation occurred in the parC gene, and were recognized as wild-type strains. The other 23 strains (19.2%) exhibited resistance to ciprofloxacin, which have A to C transversion at nucleotide 238 in the parC gene and were recognized as A238C mutant strains (shown in Table 5). After amplification of the parC gene using parC202 and parC348 specific primers, a 147 bp PCR product was obtained. The parC238 probe set (comprising probes parC238-FL and parC238-LC640) was hybridized with the single strand of said product, followed by performing the melting point (Tm) analysis of the hybrid. The Tms of the wild-type strains are 47.4±0.5° C. (CV % of 1.16), and the Tms of the mutants belonging to A238C mutation type are 61.4±0.3° C. (CV % of 0.47) (shown in Table 5 and FIG. 2). The results indicated that the parC238 probe can efficiently and rapidly distinguish the wild-type Salmonella strains from the strains having a single point mutation in the parC gene.

TABLE 4
Tms of gyrA4 hybridization probes hybridized with single-stranded PCR
products of Salmonella gyrA gene.

| | Number of | Susceptibility^{a} | | Mutation point | | | Amino acid | | Tm ± SD (CV %) |
|---|---|---|---|---|---|---|---|---|---|
| Strain type | strain (%) | NA | Cp | 248^{b} | 259 | 260 | 83^{c} | 87 | gyrA4^{d} |
| Wild type | 45 (37.5) | S | S | C | G | A | Ser | Asp | 63.6 ± 1.0 (1.62) |
| Mutant | | | | | | | | | |
| Single point | 52 (43.3) | R | S | | | | | | 59.2 ± 0.5 (0.89) |
| mutation | | | | | | | | | |
| G259A | 14 (11.7) | R | S | C | A | A | Ser | Asn | 59.8 ± 0.4 (0.60) |
| G259T | 2 (1.7) | R | S | C | T | A | Ser | Tyr | 59.6 ± 0.4 (0.68) |
| C248A | 11 (9.2) | R | S | A | G | A | Tyr | Asp | 58.6 ± 0.3 (0.51) |
| C248T | 25 (20.8) | R | S | T | G | A | Phe | Asp | 59.1 ± 0.2 (0.40) |
| Double point | | | | | | | | | |
| mutation | | | | | | | | | |
| C248T/A260G | 23 (19.2) | R | R | T | G | G | Phe | Gly | 54.6 ± 0.2 (0.27) |

^{a}NA, nalidixic acid; Cp, ciprofloxacin; R, resistance; S, susceptibility.
^{b}position of nucleotide.
^{c}position of amino acid.
^{d}hybridization probe in Table 1.

TABLE 5
Tms of parC mutation probes hybridized with single-stranded
PCR products of parC gene

| | | Number | | Amino | |
|---|---|---|---|---|---|
| | | of | Mutation | acid | Tm ± SD |
| Strain | Ciprofloxacin | strain | point | (codon) | (CV %) |
| type | susceptibility | (%) | 238^{a} | 80^{b} | parC238^{c} |
| Wild type | Sensitive | 97 | A | Ser (AGC) | 47.4 ± 0.5 |
| | | (80.8) | | | (1.16) |
| Mutant | Resistant | 23 | C | Arg | 61.4 ± 0.3 |
| (A238C) | | (19.2) | | (CGC) | (0.47) |

^{a}position of nucleotide.
^{b}position of amino acid.
^{c}hybridization probes in Table 1.

From the study results of the mechanism of the resistance of Salmonella spp. to quinolone antibacterials using the oligonucleotide primers of the present invention, the bacterial strains exhibited high resistance to nalidixic acid (MICs≧256 μg/ml) which was primarily associated with the occurrence of point mutations in the QRDR region of the gyrA gene in said strains. The main mutation positions are Ser83 and Asp87. Furthermore, the point mutations in the parC gene can promote the bacterial strains to produce high resistance to ciprofloxacin (MICs 4˜≧32 μg/ml). However, mutation in the parC gene was not found in the strains which are resistant to nalidixic acid and susceptible to ciprofloxacin. Among the 120 Salmonella strains tested, five different types of mutation are present in gyrA of 75 strains resistant to nalidixic acid, in which 23 strains also showed resistance to ciprofloxacin. Namely, in addition to point mutations in gyrA, the 23 strains also simultaneously had one point mutation in parC.

The amplification of gyrA using specific primers gyrA55 and gyrA330 designed based on the gyrA gene resulted in a 276 bp product. The amino acids translated from the nucleotides of said region are Ile29 to Gly110, which cover the QRDR region between Ala67 and Tyr122, indicating that the primers of the present invention can amplify target Ser83 and Asp87 regions of gyrA in which mutations most commonly occur. As a result, after the probe sets designed based on the types of mutation present in the genes corresponding to Ser83 and Asp87, which can specifically bind to the 276 bp product, were further used followed by the melting curve analysis, different Tms of the strains can be detected due to change of binding ability of the product to the hybridization probes, resulting from the single point mutation or double point mutation in the genes of the mutants, whereby the mutants of different mutation types can be distinguished.

In the several probe sets of the present invention which were designed based on the gyrA genes in different serotypes of Salmonella strains collected from animal products and clinical samples, the gyrA4 probe set can rapidly differentiate wild-type strains from mutants having single or double point mutations. As the 75 Salmonella strains resistant to nalidixic acid had single or double point mutations in the gyrA gene, and the strains having double point mutations also showed resistance to ciprofloxacin, said probe set can rapidly detect the bacterial strains having resistance to nalidixic acid and/or ciprofloxacin. According to the test results, the gyrA4 probe set can be applied to not only rapidly detect Salmonella strains resistant to nalidixic acid and/or ciprofloxacin but also preliminarily screen the detected strains for wild-type strains or mutant strains having single or double point mutations. Furthermore, application of real-time PCR for amplification combined with the melting curve analysis using the probe sets designed for the present invention to detect Salmonella spp. resistant to quinolone antibacterials can completely distinguish the mutation types occurred in the gyrA gene. Namely, the results indicated that said probe sets are valuable to be further applied in epidemiologic research.

Moreover, using a specific probe set, parC202 and parC348, designed based on the parC gene, the amino acids translated from the 147 bp amplified product fragment were Gly68 to Ala116 which cover the Ser80 region of parC in the 120 Salmonella strains. The probe set parC238 designed based on nucleotide 238 C of parC in the mutants resistant to ciprofloxacin can bind to the 147 bp single-stranded product. In the Tm analysis of the 97 wild-type ciprofloxacin-sensitive Salmonella strains, nucleotide 238 in parC is A, thereby changing the binding ability of said region of the single-stranded PCR product to the hybridization probes. Thus, the measured Tms of the wild-type strains were lower than those of the drug resistant strains, whereby the wild-type strains can be rapidly differentiated from those resistant to ciprofloxacin.

We claim:

1. A method for rapidly detecting Salmonella spp. resistant to quinolone antibacterials, which comprises the steps of:
(a) isolating Salmonella DNA from a sample to be detected;
(b) amplifying the Salmonella DNA obtained from step (a) by polymerase chain reaction (PCR) using a primer set derived from the nucleotide sequences of gyrA or parC gene;
(c) hybridizing a probe set based on the nucleotide sequences of the gyrA or parC gene with the single-stranded PCR product obtained from step (b); and
(d)performing melting curve analysis to analyze the Tm change of the hybrid of the single-stranded PCR product with the hybridization probes, thereby distinguishing wild-type Salmonella strains from mutants resistant to quinolone antibacterials.

2. A method according to claim 1, wherein the quinolone antibacterials include nalidixic acid and ciprofloxacin.

3. A method according to claim 1, wherein the probe set is designed based on, as the detection targets, nucleotides 248, 259 and 260 of gyrA and nucleotide 238 of parC in which point mutations occur.

4. A method according to claim 1, wherein the primer set is gyrA55/gyrA330 (SEQ ID NOS:1 and 2) or parC202/parC348 (SEQ ID NOS:3 and 4).

5. A method according to claim 3, wherein the probe set is the gyrA4 probe set (SEQ ID NOS: 5 and 6) or the parC238 probe set (SEQ ID NOS: 7 and 8).

6. A method according to claim 4, wherein the primer set gyrA55/gyrA330 (SEQ ID NOS:1 and 2) is used in combination with the gyrA4 probe set (SEQ ID NOS:5 and 6) to distinguish wild-type Salmonella strains from mutants having single or double point mutations in gyrA.

7. A method according to claim 6, which is used to detect Salmonella strains resistant to nalidixic acid and/or ciprofloxacin.

8. A method according to claim 4, wherein the primer set parC202/parC348 (SEQ ID NOS:3 and 4) is used in combination with the parC238 probe set (SEQ ID NOS:7 and 8) to distinguish wild-type Salmonella strains from mutants having single point mutations in parC.

9. A method according to claim 8, which is used to detect Salmonella strains resistant to ciprofloxacin.

10. A method according to claim 1, wherein in step (c) the probe set utilized in hybridization is used in combination with fluorescent labels.

11. A method according to claim 10, wherein one probe is labeled with fluorescent LC-Red640 and the other probe assists to produce fluorescence by fluorescence resonance energy transfer (FRET).

12. A method for rapidly detecting Salmonella spp. resistant to nalidixic acid and/or ciprofloxacin, which comprises the steps of:
(a) isolating Salmonella DNA from a sample to be detected;
(b) amplifying the Salmonella DNA obtained from step (a) by polymerase chain reaction (PCR) using the primer set gyrA55/gyrA330 (SEQ ID NOS:1 and 2);
(c) hybridizing the probe set gyrA4 (SEQ ID NOS:5 and 6) with the single-stranded PCR product obtained from step (b); and
(d) performing melting curve analysis to analyze the Tm change of the hybrid of the single-stranded PCR product with the hybridization probes, thereby distinguishing wild-type Salmonella strains from mutants having single or double point mutations in gyrA.

13. A method for rapidly detecting Salmonella spp. resistant to ciprofloxacin, which comprises the steps of:
(a) isolating Salmonella DNA from a sample to be detected;
(b) amplifying the Salmonella DNA obtained from step (a) by polymerase chain reaction (PCR) using the primer set parC202/parC348 (SEQ ID NOS:3 and 4);
(c) hybridizing the probe set parC238 (SEQ ID NOS:7 and 8) with the single-stranded PCR product obtained from step (b); and
(d) performing melting curve analysis to analyze the Tm change of the hybrid of the single-stranded PCR product with the hybridization probes, thereby distinguishing wild-type Salmonella strains from mutants having single point mutations in parC.

14. An oligonucleotide primer set, wherein the first primer has the nucleotide sequence AGC TCC TAT CTG GAT TAT GC (gyrA55, SEQ ID NO:1) and the second primer has the nucleotide sequence ACC GAA GTT ACC CTG AC (gyrA330, SEQ ID NO:2).

15. An oligonucleotide primer set according to claim 14, which is used for amplifying the DNA sequence of the target region of Salmonella gyrA or the complementary strand thereof.

16. An oligonucleotide primer set according to claim 14, which is used to distinguish wild-type Salmonella strains from mutants having single or double point mutations in gyrA.

17. An oligonucleotide primer set according to claim 14, which is used to differentiate wild-type Salmonella strains from mutants resistant to nalidixic acid and/or ciprofloxacin.

18. An oligonucleotide primer set, wherein the first primer has the nucleotide sequence GGT GAC GTA CTG GGT A (parC202, SEQ ID NO:3) and the second primer has the nucleotide sequence CGC GAA TGA CTT CGG A (parC348, SEQ ID NO:4).

19. An oligonucleotide primer set according to claim 18, which is used for amplifying the DNA sequence of the target region of Salmonella parC or the complementary strand thereof.

20. An oligonucleotide primer set according to claim 18, which is used to distinguish wild-type Salmonella strains from mutants having single point mutations in parC.

21. An oligonucleotide primer set according to claim 18, which is used to differentiate wild-type Salmonella strains from mutants resistant to ciprofloxacin.

22. An oligonucleotide probe set, wherein the first probe has the nucleotide sequence CGA TTC CGC AGT GTA TGA CAC C-FL (gyrA4-FL, SEQ ID NO:5) and the second probe has the nucleotide sequence LCRED640-CGT TCG TAT GGC GCA GCC ATT CTC G-PHO (gyrA4-LC640, SEQ ID NO:6).

23. An oligonucleotide probe set according to claim 22, which hybridizes with the DNA sequence of the target region of Salmonella gyrA or the complementary strand thereof.

24. An oligonucleotide probe set according to claim 22, which is used to distinguish wild-type Salmonella strains from mutants having single or double point mutations in gyrA.

25. An oligonucleotide probe set according to claim 22, which is used for detection of Salmonella mutant strains resistant to nalidixic acid and/or ciprofloxacin.

26. An oligonucleotide probe set, wherein the first probe has the nucleotide sequence CGA CCG CGC CTG CT-FL (parC238-FL, SEQ ID NO:7) and the second probe has the nucleotide sequence LCRED640-GAA GCC ATG GTG CTG ATG GCG-PHO (parC238-LC640, SEQ ID NO:8).

27. An oligonucleotide probe set according to claim 26, which hybridizes with the target region of Salmonella parC or the complementary strand thereof.

28. An oligonucleotide probe set according to claim 26, which is used to distinguish wild-type Salmonella strains from mutants having single point mutations in parC.

29. An oligonucleotide probe set according to claim 26, which is used for detection of Salmonella mutant strains resistant to ciprofloxacin.


SEQUENCE LISTING (8 sequences)

SEQ ID NO: 1 - DNA, 20 bp, Salmonella sp.
agctcctatctggattatgc

SEQ ID NO: 2 - DNA, 17 bp, Salmonella sp.
accgaagttaccctgac

SEQ ID NO: 3 - DNA, 16 bp, Salmonella sp.
ggtgacgtactgggta

SEQ ID NO: 4 - DNA, 16 bp, Salmonella sp.
cgcgaatgacttcgga

SEQ ID NO: 5 - DNA, 22 bp, Salmonella sp.
cgattccgcagtgtatgacacc

SEQ ID NO: 6 - DNA, 25 bp, Salmonella sp.
cgttcgtatggcgcagccattctcg

SEQ ID NO: 7 - DNA, 14 bp, Salmonella sp.
cgaccgcgcctgct

SEQ ID NO: 8 - DNA, 21 bp, Salmonella sp.
gaagccatggtgctgatggcg